(19) United States
(12) Patent Application Publication
(10) Pub. No.: US 2024/0366311 A1
(43) Pub. Date: Nov. 7, 2024

(54) MEDICAL INSTRUMENT POSITION SENSOR SLEEVE WITH INTEGRAL HEATING ELEMENT

(71) Applicants: Acclarent, Inc., Irvine, CA (US); Biosense Webster (Israel) Ltd., Yokneam (IL)

(72) Inventors: Raymond Yue-sing Tang, Rosemead, CA (US); Gigi Koe, Anaheim, CA (US)

(21) Appl. No.: 18/594,164

(22) Filed: Mar. 4, 2024

Related U.S. Application Data
(60) Provisional application No. 63/463,327, filed on May 2, 2023.

(51) Int. Cl. A61B 34/20; A61B 17/00; A61B 17/32

(52) U.S. Cl. CPC A61B 34/20; A61B 17/00234; A61B 17/32002; A61B 2034/2051; A61B 2217/005

(57) ABSTRACT

An apparatus for use with an ENT instrument includes a shaft, a heat shrink material, and a heating element. The shaft defines a hollow interior. The hollow interior is configured to receive a tubular portion of the ENT instrument. The heat shrink material is integrated into a portion of the shaft. The heating element is fixedly secured to the shaft or the heat shrink material. The heating element is configured to heat a portion of the heat shrink material to reduce a diameter of a portion of the shaft at a predetermined position along the length of the shaft.

### PRIORITY

This application claims priority to U.S. Provisional Pat. App. No. 63/463,327, entitled “Medical Instrument Position Sensor Sleeve with Integral Heating Element,” filed May 2, 2023, the disclosure of which is incorporated by reference herein, in its entirety.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation system that may be used in IGS procedures is the CARTO® 3 System by Biosense-Webster, Inc., of Irvine, California. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, some instruments can include sensors (e.g., electromagnetic coils that emit electromagnetic fields and/or are responsive to externally generated electromagnetic fields), which can be used to perform the procedure while the sensors send data to the computer indicating the current position of each sensor-equipped instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

In some instances, it may be desirable to guide an instrument using IGS techniques described above. However, certain IGS navigation systems may be equipped for use with specific instruments and certain instruments may be equipped for use with specific IGS navigation systems or no IGS navigation system at all. Thus, it may be desirable to provide an easily implemented and cost-effective means to incorporate compatibility with a given IGS navigation system into a variety of instruments. While several systems and methods have been made and used in connection with IGS navigation stems, it is believed that no one prior to the inventors has made or used the invention described in the appended claims.

### BRIEF DESCRIPTION OF THE DRAWINGS

The drawings and detailed description that follow are intended to be merely illustrative and are not intended to limit the scope of the invention as contemplated by the inventors.

FIG. 1 depicts a schematic view of an example of a surgery navigation system being used on a patient seated in an example of a medical procedure chair;

FIG. 2 depicts a perspective view of an example of an instrument with a navigation adapter sheath removably secured to the instrument;

FIG. 3 depicts a perspective view of the instrument and navigation adapter sheath of FIG. 2, with the navigation adapter sheath removed from the instrument;

FIG. 4A depicts a perspective exploded view of the navigation adapter sheath of FIG. 2;

FIG. 4B depicts a detailed top plan view of the navigation adapter sheath of FIG. 4B;

FIG. 5 depicts a top plan view of a flexible navigation sensor assembly of the navigation adapter sheath of FIG. 2, the flexible navigation sensor assembly in a flat configuration;

FIG. 6 depicts a top plan view of an intermediate portion of the flexible navigation sensor assembly of FIG. 5 in the flat configuration;

FIG. 7 depicts a top plan view of a distal portion of the flexible navigation sensor assembly of FIG. 5 in the flat configuration;

FIG. 8A depicts a perspective series view of the navigation adapter sheath of FIG. 2 with the instrument of FIG. 2, the navigation adapter sheath being slid onto the instrument;

FIG. 8B depicts another perspective series view of the navigation adapter sheath of FIG. 2 with the instrument of FIG. 2, the navigation adapter sheath being transformed from a loose configuration to a tight configuration;

FIG. 8C depicts yet another perspective series view of the navigation adapter sheath of FIG. 2 with the instrument of FIG. 2, the navigation adapter sheath being secured to the instrument;

FIG. 9 depicts a perspective view of anther navigation adapter sheath for use with the instrument of FIG. 2;

FIG. 10A depicts a perspective series view of the navigation adapter sheath of FIG. 9 with the instrument of FIG. 2, the navigation adapter sheath being slid onto the instrument;

FIG. 10B depicts another perspective series view of the navigation adapter sheath of FIG. 9 with the instrument of FIG. 2, the navigation adapter sheath being transformed from a loose configuration to a tight configuration; and

FIG. 10C depicts yet another perspective series view of the navigation adapter sheath of FIG. 9 with the instrument of FIG. 2, the navigation adapter sheath being secured to the instrument.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical instrument having a distal surgical end effector. The term “proximal” refers to the position of an element arranged closer to the surgeon, and the term “distal” refers to the position of an element arranged closer to the surgical end effector of the surgical instrument and further away from the surgeon. Moreover, to the extent that spatial terms such as “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

As used herein, the terms “about” and “approximately” for any numerical values or ranges indicate a suitable dimensional tolerance that allows the part or collection of components to function for its intended purpose as described herein.

### I. Example of an Image Guided Surgery Navigation System

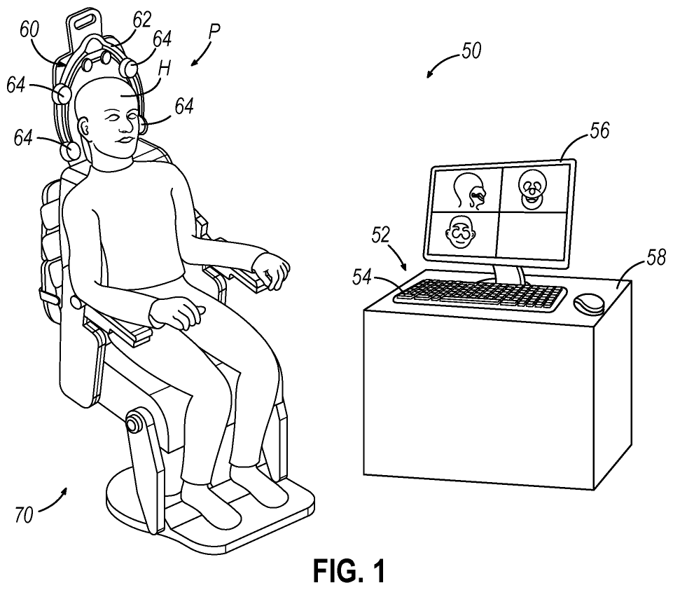

When performing a medical procedure within a head (H) of a patient (P), it may be desirable to have information regarding the position of an instrument within the head (H) of the patient (P), particularly when the instrument is in a location where it is difficult or impossible to obtain an endoscopic view of a working element of the instrument within the head (H) of the patient (P). FIG. 1 shows an example of an IGS navigation system (50) enabling an ENT procedure to be performed using image guidance. In addition to or in lieu of having the components and operability described herein IGS navigation system (50) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein, in its entirety; and U.S. Pat. Pub. No. 2014/0364725, entitled “Systems and Methods for Performing Image Guided Procedures within the Ear, Nose, Throat and Paranasal Sinuses,” published Dec. 11, 2014, now abandoned, the disclosure of which is incorporated by reference herein, in its entirety.

IGS navigation system (50) of the present example comprises a field generator assembly (60), which comprises set of magnetic field generators (64) that are integrated into a horseshoe-shaped frame (62). Field generators (64) are operable to generate alternating magnetic fields of different frequencies around the head (H) of the patient (P). An instrument, such as any of the instruments described below, may be inserted into the head (H) of the patient (P). Such an instrument may be a standalone device or may be positioned on an end effector. In the present example, frame (62) is mounted to a chair (70), with the patient (P) being seated in the chair (70) such that frame (62) is located adjacent to the head (H) of the patient (P). By way of example only, chair (70) and/or field generator assembly (60) may be configured and operable in accordance with at least some of the teachings of U.S. Pat. No. 10,561,370, entitled “Apparatus to Secure Field Generating Device to Chair,” Issued Feb. 18, 2020, the disclosure of which is incorporated by reference herein, in its entirety.

IGS navigation system (50) of the present example further comprises a processor (52), which controls field generators (64) and other elements of IGS navigation system (50). For instance, processor (52) is operable to drive field generators (64) to generate alternating electromagnetic fields; and process signals from the instrument to determine the location of a navigation sensor or position sensor in the instrument within the head (H) of the patient (P). Processor (52) comprises a processing unit (e.g., a set of electronic circuits arranged to evaluate and execute software instructions using combinational logic circuitry or other similar circuitry) communicating with one or more memories. Processor (52) of the present example is mounted in a console (58), which comprises operating controls (54) that include a keypad and/or a pointing device such as a mouse or trackball. A physician uses operating controls (54) to interact with processor (52) while performing the surgical procedure.

While not shown, the instrument may include a navigation sensor or position sensor that is responsive to positioning within the alternating magnetic fields generated by field generators (64). A coupling unit (not shown) may be secured to the proximal end of the instrument and may be configured to provide communication of data and other signals between console (58) and the instrument. The coupling unit may provide wired or wireless communication of data and other signals.

In some versions, the navigation sensor or position sensor of the instrument may comprise at least one coil at or near the distal end of the instrument. When such a coil is positioned within an alternating electromagnetic field generated by field generators (64), the alternating magnetic field may generate electrical current in the coil, and this electrical current may be communicated along the electrical conduit(s) in the instrument and further to processor (52) via the coupling unit. This phenomenon may enable IGS navigation system (50) to determine the location of the distal end of the instrument within a three-dimensional space (i.e., within the head (H) of the patient (P), etc.). To accomplish this, processor (52) executes an algorithm to calculate location coordinates of the distal end of the instrument from the position related signals of the coil(s) in the instrument. Thus, a navigation sensor may serve as a position sensor by generating signals indicating the real-time position of the sensor within three-dimensional space.

Processor (52) uses software stored in a memory of processor (52) to calibrate and operate IGS navigation system (50). Such operation includes driving field generators (64), processing data from the instrument, processing data from operating controls (54), and driving display screen (56). In some implementations, operation may also include monitoring and enforcement of one or more safety features or functions of IGS navigation system (50). Processor (52) is further operable to provide video in real time via display screen (56), showing the position of the distal end of the instrument in relation to a video camera image of the patient's head (H), a CT scan image of the patient's head (H), and/or a computer-generated three-dimensional model of the anatomy within and adjacent to the patient's nasal cavity. Display screen (56) may display such images simultaneously and/or superimposed on each other during the surgical procedure. Such displayed images may also include graphical representations of instruments that are inserted in the patient's head (H), such that the operator may view the virtual rendering of the instrument at its actual location in real time. By way of example only, display screen (56) may provide images in accordance with at least some of the teachings of U.S. Pat. No. 10,463,242, entitled “Guidewire Navigation for Sinuplasty,” issued Nov. 5, 2019, the disclosure of which is incorporated by reference herein, in its entirety. In the event that the operator is also using an endoscope, the endoscopic image may also be provided on display screen (56).

The images provided through display screen (56) may help guide the operator in maneuvering and otherwise manipulating instruments within the patient's head (H). It should also be understood that other components of a surgical instrument and other kinds of surgical instruments, as described below, may incorporate a navigation sensor like the navigation sensor described above.

II. Example of a Navigation Adapter Sheath for Use with One or More Surgical Instruments

In some instances, it may be desirable to provide an adapter with a flex circuit having one or more integral navigation sensors, where such an adapter may be readily coupled with an instrument that otherwise lacks any navigation sensors. In such a scenario, the adapter may impart navigation capabilities to the instrument. Similarly, it may be desirable to use an adapter with a flex circuit having one or more integral navigation sensors in combination with an instrument that already has one or more navigation sensors, where the position data from the one or more navigation sensors of the adapter may supplement the position data from the one or more navigation sensors of the instrument. In such a scenario, the adapter may enhance navigation capabilities to the instrument. In either of the above scenarios, the adapter may be configured to avoid adding bulk to the instrument; and to be easily assembled with the instrument in the surgical field or operating room. Moreover, the adapter may be configured to accommodate different kinds of instruments and/or instruments otherwise having different outer diameters, such that the adapter need not necessarily be limited in its compatibility to just one single instrument size or type.

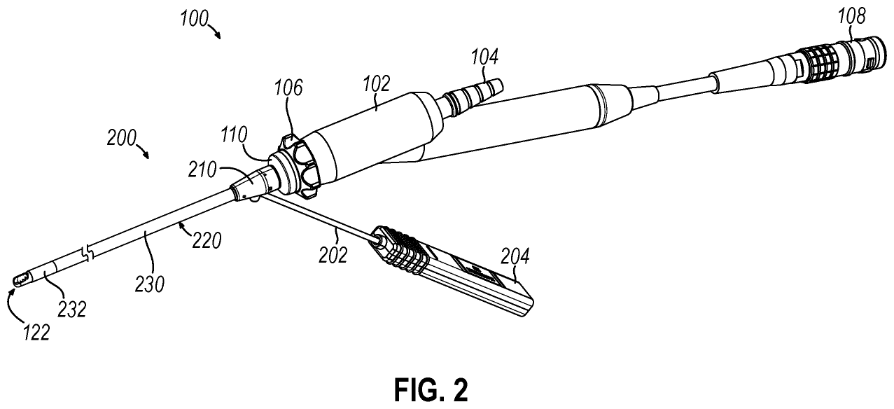

FIGS. 2-4B show an example of an adapter, that may provide the above-described benefits and functionality, in the form of an adapter sheath (200). In the present example, adapter sheath (200) is coupled with a tissue shaving instrument (100). Tissue shaving instrument (100) of this example includes a body (102), a suction port (104), a rotation control knob (106), a power connector (108), a hub (110), and a shaft assembly (120). Body (102) is configured for grasping by an operator. Suction port (104) is configured to couple with a suction source, such that suction may be applied to draw tissue and fluids during operation of tissue shaving instrument (100). Rotation control knob (106) is rotatable relative to body (102) to reorient shaft assembly (120) about the longitudinal axis of shaft assembly (120). Power connector (108) is configured to couple with a power source to provide power to a motor (not shown) in body (102). The motor is operable to drive a cutting shaft (not shown) of shaft assembly (120) in a manner as is known in the art.

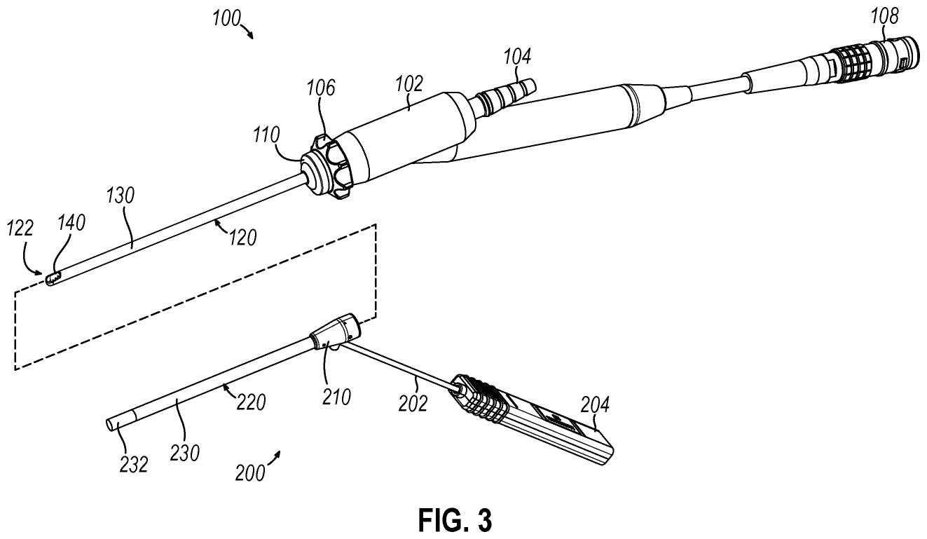

In addition to including a cutting shaft, shaft assembly (120) includes an outer shaft (130) and an end effector (122). End effector (122) is at the distal end of shaft assembly (120). As best seen in FIG. 3, end effector (122) includes a transverse opening (140) formed at the distal end of outer shaft (130). Optionally, transverse opening (140) may include a serrated cutting edge in some examples extending along the perimeter of opening (140). The cutting shaft is positioned inside outer shaft (130) and includes a similar transverse opening and cutting edge that complement transverse opening (140) and the cutting edge of outer shaft (130). The motor in body (102) drives rotation of the cutting shaft relative to outer shaft (130), about the longitudinal axis of shaft assembly (120). During such rotation, suction is applied via the lumen defined by the cutting shaft to draw tissue into opening (140), the cutting edge of the cutting shaft cooperates with the cutting edge of outer shaft (130) to shear the tissue, and the sheared tissue is drawn proximally through the lumen of the cutting shaft under the influence of the suction. Those skilled in the art will understand that it may be beneficial to have data from navigation sensors indicating the real-time location of end effector (122) within the patient (P) during operation of tissue shaving instrument (100).

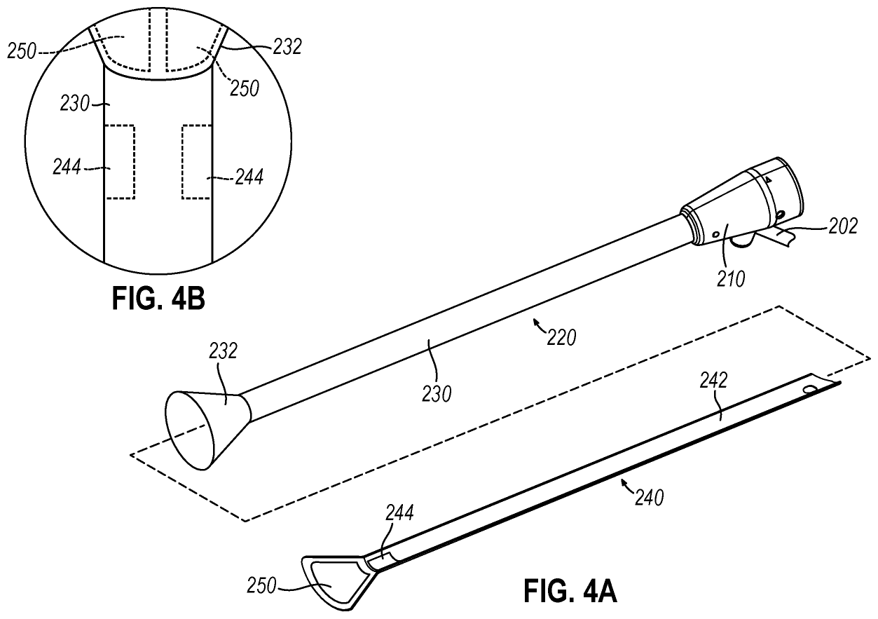

As shown in FIGS. 3-4B, adapter sheath (200) of the present example includes a hub (210), a shaft assembly (220) extending distally from hub (210), a cable (202), and a connector (204). As best seen in FIG. 4A, shaft assembly (220) includes a hollow outer shaft (230) and a flex circuit (240). Hub (210) is fixedly secured to the proximal end of outer shaft (230).

Outer shaft (230) includes an attachment portion (232) disposed proximate the distal end of outer shaft (230). Attachment portion (232) is configured to transform in shape in the presence of heat. In particular, attachment portion (232) may begin in a pre-determined loose configuration for insertion of shaft assembly (120) of tissue shaving instrument (100) into outer shaft (230). Upon the application of heat, attachment portion (232) may transform to a tight configuration where attachment portion (232) is tight against the outer surface of shaft assembly (120) of tissue shaving instrument (100). In the present example, attachment portion (232) may initially be in a flared or conical configuration as shown in FIG. 4A. Such a flared or conical configuration is merely optional and, in other versions, attachment portion (232) may merely define a cylindrical shape with an oversized diameter relative to the diameter of out shaft (130) of tissue shaving instrument (100).

As will be described in greater detail below, attachment portion (232) is formed of a material responsive to heat or other environmental stimuli. For instance, in the present example, attachment portion includes a low temperature heat shrink material that is configured to shrink in response to the presence of heat. Of course, other suitable materials may be used such as shape memory alloys, stimuli-sensitive polymers, and/or etc. In some examples, the material of attachment portion (232) is distinct from other materials of outer shaft (230). In other examples, the material of attachment portion (232) may be of a substantially similar material as the rest of outer shaft (230).

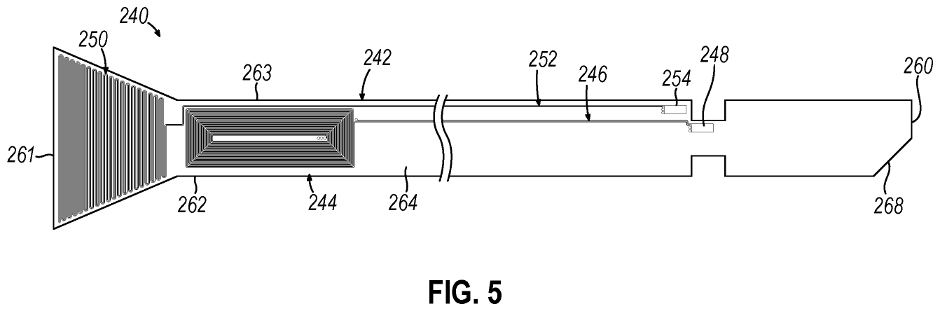

Flex circuit (240) includes a flexible substrate (242), one or more navigation sensors (244), one or more heating elements (250), and traces (246, 252) (see FIG. 5) in communication with navigation sensors (244) and heating elements (250). As will be described in greater detail below, flexible substrate (242), navigation sensors (244), and traces (246) may be configured to interact with IGS navigation system (50) to locate one or more portions of adapter sheath (200) relative to a patient. Flex circuit (240) may have just one single layer or a plurality of layers.

Flex circuit (240) of the present example includes one heating element (250). Thus, when combined with another flex circuit (240), multiple heating elements (250) may be positioned within shaft assembly (220). Each heating element (250) is generally configured to selectively emit heat. By way of example only, each heating element (250) may be configured as a resistive heating element. As will be described in greater detail below, heat emitted by heating elements (250) may be used to control the shape and size of outer shaft (230) via interaction with attachment portion (232). Additionally, in some examples, heating elements (250) may optionally be configured to function similarly to navigation sensors (244) when not emitting heat. In some such examples, heating elements (250) may form coils that generate electrical current in response to alternating magnetic fields from field generators (64), withs such electrical signals from heating elements (250) being indicative of the respective positions of heating elements (250) in three-dimensional space.

Although only a single flex circuit (240) is shown in FIG. 4A, it should be understood that adapter sheath (200) may be equipped with two or more flex circuits (240) to provide multiple navigation sensors (244) and multiple heating elements (250). For instance, as seen in FIG. 4B, the present example includes two flex circuits (240) positioned on opposite sides of outer shaft (230) to provide two opposing navigation sensors (244) and two opposing heating elements (250). Alternatively, two or more navigation sensors (244), two or more heating elements (250) or various combinations thereof may be placed on a single flex circuit (240) in some examples.

Hub (210) of adapter sheath (200) is configured to engage with hub (110) of tissue shaving instrument (100) to thereby removably secure a proximal end of adapter sheath (200) relative to tissue shaving instrument (100). By way of example only, hub (210) may provide a snap fit, and interference fit, or any other suitable kind of relationship with hub (110). It should be understood that in some versions, such engagement between hub (210) and hub (110) is entirely optional and may be omitted. For instance, in some examples, adapter sheath (200) may be used with a variety of instruments having different shaft lengths. In such examples, it may still be desirable position features of adapter sheath (200) (e.g., navigation sensors (244) and/or heating elements (250)) proximate to specific features of such instruments (e.g., a distal end or end effector). Thus, it may be desirable for the position of adapter sheath (200) relative to the shaft of such instruments to not be tied to particular structural features such as a hub similar to hub (110). In other words, in some examples, a gap may be present between hub (110) and hub (210). It is therefore not essential that hub (210) of adapter sheath (200) contact or otherwise engage hub (110) of tissue shaving instrument (100). Moreover, some variations of adapter sheath (200) may lack hub (110).

As shown in FIG. 2, the length of shaft assembly (220) is such that the distal end of outer shaft (230) is just proximal to end effector (122) when adapter sheath (200) is fully seated on tissue shaving instrument (100). Thus, navigation sensors (244) and heating elements (250) of adapter sheath (200) are positioned proximate to end effector (122). Such a location of navigation sensors (244) and heating elements (250) may thus allow navigation sensors (244) to readily indicate the real-time position of end effector (122), as will be described in greater detail below. Furthermore, such a position of heating elements (250) may permit heating elements (250) to be in thermal communication with attachment portion (232) such that heat may be communicated from heating elements (250) to attachment portion (232).

Traces (246) of flex circuit (240) are configured to communicate position-indicative signals from navigation sensors (244) to cable (202). Additionally, traces (252) are configured to communicate current to heating elements (250) to generate heat via heating elements (250). Optionally, traces (252) may also be configured to communication position-indicative signals from heating elements (250). Cable (202) is configured to communicate these position-indicative signals to connector (204) and receive electric current for heating from connector (204). Connector (204) is configured to couple with IGS navigation system (50) and thereby communicate the position-indicative signals to IGS navigation system (50). In some versions, connector (204) is configured to plug into a corresponding socket of IGS navigation system (50). Some versions of IGS navigation system (50) may also provide power to heating elements (250) via connector (204). Alternatively, some variations of adapter sheath (200) may provide one cable and/or connecter between heating elements (250) and a source of power for heating elements and another separate cable and/or connecter coupling between navigation sensors (244) and IGS navigation system (50).

In some other versions, connector (204) includes a wireless transmitter that is operable to wirelessly transmit the position-indicative signals IGS navigation system (50). In still other versions, cable (202) and connector (204) are omitted, and some other component of adapter sheath (200) is configured to wirelessly transmit the position-indicative signals IGS navigation system (50). By way of example only, one or more wireless transmitters may be integrated into hub (210). Alternatively, the position-indicative signals may be communicated to IGS navigation system (50) in any other suitable fashion.

While adapter sheath (200) is shown and described in the context of a tissue shaving instrument (100), adapter sheath (200) may be readily used with any other suitable kind of instrument. Adapter sheath (200) need not necessarily be limited to the context of tissue shaving instruments like tissue shaving instrument (100). By way of example only, adapter sheath (200) may be configured to fit over endoscopes, suction cannulas, various kinds of ENT instruments, and/or any other kind of instrument as will be apparent to those skilled in the art in view of the teachings herein. The length of adapter sheath (200) may vary based on the kind of instrument with which adapter sheath (200) will be coupled. Regardless of the kind of instrument with which adapter sheath (200) is coupled, adapter sheath (200) may be readily coupled with the instrument right before the medical procedure in which the instrument will be used with adapter sheath (200). In other words, adapter sheath (200) may be coupled with the instrument in the operating room, physician's office, or other room/facility in which the medical procedure will take place. In some scenarios, after the medical procedure is complete, adapter sheath (200) may be removed from the instrument. The removed adapter sheath (200) may be disposed of or be sterilized for subsequent reuse. In some other scenarios, adapter sheath (200) is disposed of with the instrument to which adapter sheath (200) is attached.

FIGS. 5-7 show flex circuit (240) in additional detail. Flex circuit (240) of the present example is provided in the form of a flexible printed circuit board (PCB) and includes an elongate, generally rectangular flex circuit substrate (242) with a plurality of traces (246, 252) and a plurality of corresponding trace leads (248, 254) formed (e.g., printed and/or embedded) thereon. As shown, substrate (242) extends longitudinally between proximal and distal ends (260, 261), laterally between first and second sides (262, 263), and vertically between a top surface (264) and a bottom surface (not shown). In some examples, substrate (242) may optionally include one or more through bores (not shown) extending between top surface (264) and the bottom surface. Such through bores may be configured to receive respective pins (not shown) or other suitable fasteners for securing flex circuit (240) to outer shaft (230) and/or to any other component of adapter sheath (200). In the version shown, substrate (242) also optionally includes a proximal bevel (268) extending between proximal end (260) and first side (262). Proximal bevel (268) may be configured to assist in locating proximal end (260) at a desired position relative to outer shaft (230) and/or relative to any other component of adapter sheath (200).

Substrate (242) may be formed of an electrically-insulative, flexible plastic material such as polyimide or liquid crystal polymer (LCP). For example, substrate (242) may be formed of polyimide in cases where maintaining a relatively flat configuration of substrate (242) is desired since such a substrate (242) formed of polyimide may be resiliently biased toward a naturally flat configuration. Alternatively, substrate (242) may be formed of LCP in cases where a more complex geometrical configuration and/or increased flexibility of substrate (242) is desired since a substrate (242) formed of LCP may be thermoformed to accommodate such complex geometries and/or provide increased flexibility. In any event, traces (246, 252) and leads (248, 254) may each be formed of an electrically-conductive, metallic material such as copper. Flex circuit (240) is suitably sized to fit within an interior of outer shaft (230) without obstructing the hollow interior of outer shaft (230), thereby permitting insertion of shaft assembly (120) of tissue shaving instrument (100) into outer shaft (230). In this regard, flex circuit (240) may have a relatively low profile, at least by comparison to traditional coil sensors. In some versions, flex circuit (240) may have a thickness of approximately 50 microns. While flex circuit (240) is positioned within an interior of outer shaft (230) in this example, flex circuit (240) may alternatively be positioned about the exterior of outer shaft (230) or within a sidewall region of outer shaft (230).

As best seen in FIG. 5, traces (246, 252) extend proximally from a respective navigation sensor (244) or heating element (250) to a respective trace lead (248, 254) oriented proximate proximal end (260). Traces (246, 252) of the present version are generally formed on top surface (264) of substrate (242). Each trace (246, 252) includes two separate traces (246a, 246b, 252a, 252b) running in parallel relative to each other. Optionally, in some versions, one or more of separate traces (246a, 246b, 252a, 252b) may extend laterally through substrate (242) from top surface (264) to the bottom surface (not shown). For instance, in the present version, one trace lead (248, 254) may be positioned on top surface (264), while another identical trace lead (248, 254) may be positioned on the bottom surface. A given separate trace (246a, 246b, 252a, 252b) may thus extend laterally through substrate (242) to be electrically coupled to a given trace lead (248, 254). In other versions, flex circuit (240) may be configured with one separate trace (246a, 252a) on top surface (264) and another separate trace (246b, 252b) on the bottom surface, or vice versa. In still other versions, all separate traces (246a, 246b, 252a, 252b) and corresponding trace leads (248, 254) may be isolated to a single surface.

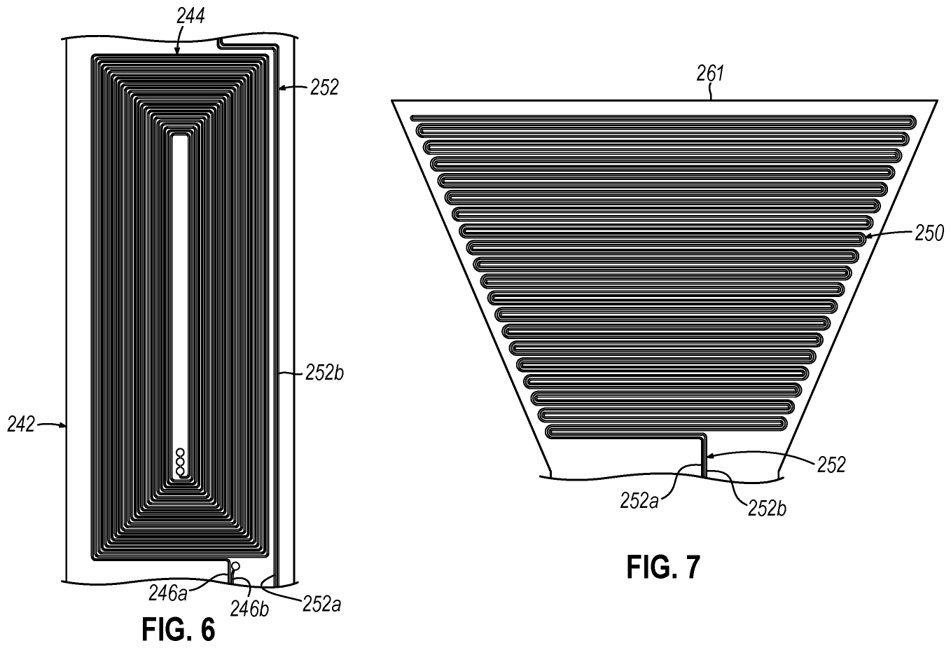

Navigation sensor (244) is shown in greater detail in FIG. 6. As can be seen, navigation sensor (244) includes a series of concentric loops of an electrically-conductive, metallic material such as copper. In this configuration, one separate lead (246a) associated with navigation sensor (244) is electrically coupled to the radially outer end of concentric loops forming navigation sensor (244), while another separate lead (246b) associated with navigation sensor (244) is electrically coupled to the radially inner end of the concentric loops forming navigation sensor (244). Additionally, a portion of separate lead (246b) may extend laterally though substrate (242) and across bottom surface (or within an internal layer of substrate (242)) to electrically couple to the radially inner end of the concentric loops forming navigation sensor (244).

The concentric loops forming navigation sensor (244) are operable to generate signals indicative of the position of navigation sensor (244) and thereby indicative of the position of at least a portion (e.g., outer shaft (230)) of adapter sheath (200) in three-dimensional space. The position data generated by such position related signals may be processed by processor (52) for providing a visual indication to the operator to show the operator where outer shaft (230) of adapter sheath (200) is located within the patient (P) in real time. Such a visual indication may be provided as an overlay on one or more preoperatively obtained images (e.g., CT scans) of the patient's anatomy, within a 3D digital model of the patient's anatomy, and/or otherwise.

Heating element (250) is shown in greater detail in FIG. 7. As can be seen, heating element (250) includes a single loop of an electrically-conductive, metallic material such as copper. This single loop includes two generally parallel traces of metallic material serpentined within an area corresponding to the shape of at least a portion of attachment portion (232). As described above, attachment portion (232) of the present example is of a flared or conical shape. Thus, heating element (250) of the present example defines a series of rectangular sections progressively increasing in length to define a generally triangular shape that may be bent into a partially conical shape corresponding to a portion of the shape of attachment portion (232). Of course, in examples where the shape of attachment portion (232) is varied, the shape of heating element (250) may likewise be varied to correspond to at least a portion of the shape of attachment portion (232).

Although heating element (250) of the present example is formed of a single loop of electrically-conductive material, it should be understood that in other examples, heating element (250) may be formed of a plurality of loops of electrically-conductive material in a variety of shapes. For instance, in some examples, heating element (250) may be formed of two separate loops, with each loop being arranged a series of rectangular sections of varying sizes to form a variety of suitable shapes. Alternatively, in another example, heating element (250) is formed of a series of concentric loops similar to the configuration of navigation sensor (244) described above. Such configurations may be desirable in circumstances where heating element (250) may be used for the secondary purpose of navigation in combination with IGS navigation system (50). Of course, other suitable configurations of heating element (250) will be apparent to those skilled in the art in view of the teachings herein.

In the example shown, navigation sensor (244) is positioned near end effector (122) of tissue shaving instrument (100) for facilitating navigation of end effector (122). Additionally, in other examples, additional navigation sensors may be included at a variety of proximal positions along outer shaft (230) for assisting in identifying the direction and/or orientation of outer shaft (130) of tissue shaving instrument (100), for example. However, it will be appreciated that navigation sensor (244) and/or other additional navigation sensors may be positioned at any other suitable locations relative to components of tissue shaving instrument (100) for which navigation is desired. Additionally, in some examples, flex circuit (240) includes one or more additional corresponding navigation sensors on the bottom surface thereof to form a navigation sensor pair with navigation sensor (244), for example. Such pairs of navigation sensor pairs may assist in improving the accuracy of location coordinates of outer shaft (230) calculated by processor (52) from the position related signals of such navigation sensor pairs.

As described above, outer shaft (230) of the present example includes two substantially similar flex circuits (240) oriented on opposite sides of outer shaft (230). In this regard, outer shaft (230) includes two navigation sensors (244) within the generally cylindrical interior of outer shaft (230) such that one navigation sensor (244) is disposed on a first lateral side of outer shaft (230) and another navigation sensor (244) is disposed on a second lateral side of outer shaft (230). In this manner, the pair of navigation sensors (244) may provide position related signals indicative of locations of both lateral sides of outer shaft (230), which may improve the accuracy of the location coordinates calculated by processor (52).

In some versions, a length of flex circuit (240) defined between proximal and distal ends (260, 261) of substrate (242) may be sufficiently great to position navigation sensor (244) near the distal end of outer shaft (230) for facilitating navigation of the distal end of outer shaft (230) while also positioning leads (248, 254) at a sufficiently proximal location whereat leads (248, 254) may be directly electrically coupled to the coupling unit (e.g., without intervening electrical wires or cables). In this regard, flex circuit (240) may have a length substantially equal to or greater than a length of outer shaft (230), such that leads (248, 254) may be positioned within or even proximally relative to hub (210) of adapter sheath (200).

While flex circuit (240) of the present example is disposed along a generally cylindrical inner surface of outer shaft (230), flex circuit (240) may alternatively be disposed along a generally cylindrical outer surface of outer shaft (230) in a generally curved configuration in which flex circuit (240) is curved about the longitudinal axis of outer shaft (230) with a radius of curvature corresponding to that of the cylindrical outer surface of outer shaft (230) to thereby conform to an outer circumference of outer shaft (230). In other examples, flex circuit (240) is integrated into a portion of outer shaft (230) itself or between two or more shafts together forming outer shaft (230). In any event, flex circuit (240) may permit space for a lumen to extend within or along outer shaft (230) for receipt of outer shaft (130) of tissue shaving instrument (100) as described above, such that flex circuit (240) may continuously communicate position related signals to processor (52) during severing and/or suctioning of tissue using tissue shaving instrument (100). In other words, severing/suctioning of tissue with outer shaft (130) of tissue shaving instrument (100) may be performed concurrently with navigation of outer shaft (230) without interfering with each other.

While not shown, flex circuit (240) may include at least one temperature sensor formed (e.g., printed and/or embedded) on substrate (242) for detecting a temperature of substrate (242) and/or the surrounding environment at or near navigation sensor (244) and/or heating element (250) and generating signals indicative of the detected temperature. The detected temperature may be processed by processor (52) for improving the accuracy of the location coordinates calculated by processor (52) from the position related signals of navigation sensors (244) and/or improving the operational characteristics of heating element (250). In other versions, changes in the temperatures of navigation sensor (244) and/or heating element (250) may be determined by detecting changes in the impedances of the respective loop portions. In any event, processor (52) may adjust the calculation of the location coordinates based on the temperature data to correct for any resistance changes caused by changes in temperature.

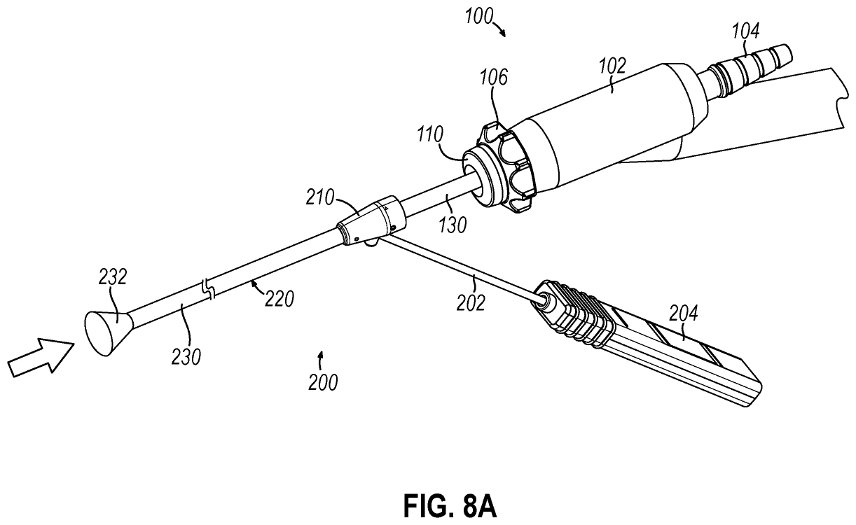

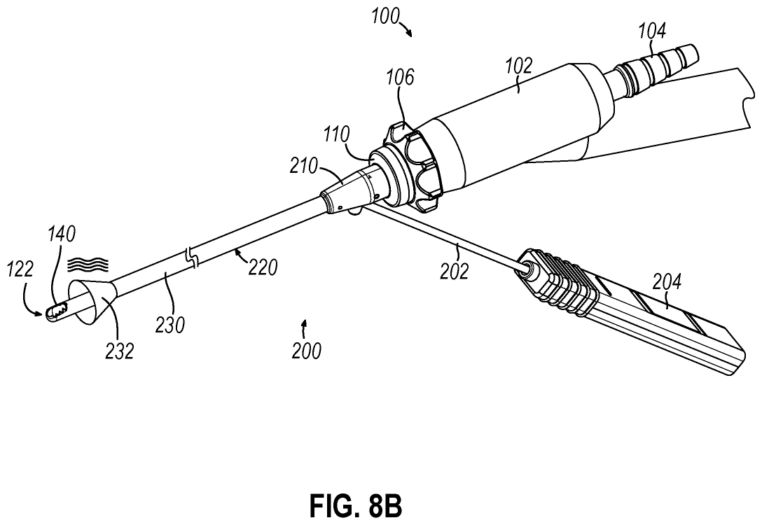

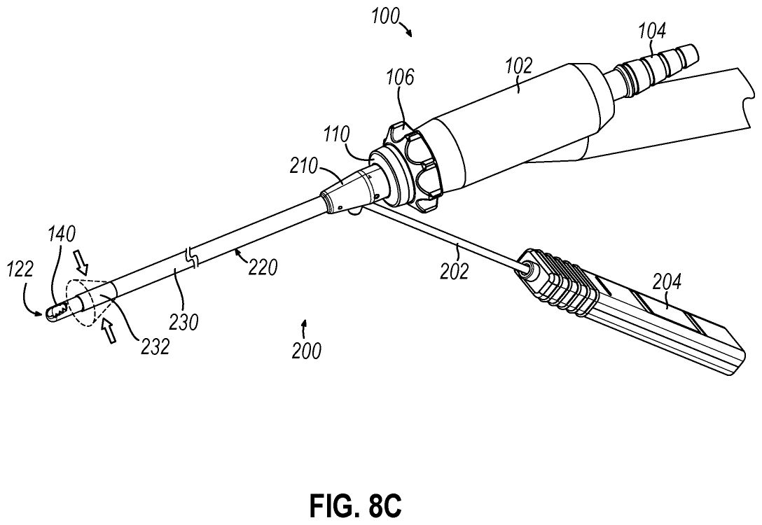

FIGS. 8A through 8C show an example method of using tissue shaving instrument (100) in combination with adapter sheath (200). Although the use shown and described herein is in the context of use with tissue shaving instrument (100), it should be understood that adapter sheath (200) may be used in combination with other suitable instruments as described above. Thus, the methods described herein may be used to adapt any suitable instrument for use with IGS navigation system (50) via adapter sheath (200).

As best seen in FIG. 8A, adapter sheath (200) may be initially inserted into shaft assembly (120) of tissue shaving instrument (100) with outer sheath (130) being inserted into the proximal end of hub (210) of adapter sheath (200). Adapter sheath (200) may then be positioned relative to outer sheath (130) of tissue shaving instrument (100) to position navigation sensors (244) and heating elements (250) at or near end effector (122) of tissue shaving instrument (100). Optionally, this may include engaging hub (110) of tissue shaving instrument (100) with hub (210) of adapter sheath (200). However, it should be appreciated that in uses with instruments other than tissue shaving instrument (100), there may be a difference between the length of the shaft assembly of such instruments and outer sheath (230). Thus, in some examples, a gap may be present between hub (210) and a corresponding hub of such alternative instruments. Such a gap may facilitate a position of navigation sensors (244) and heating elements (250) at or near an end effector or similar structures of such alternative instruments, particularly where such alternative instruments have outer diameters that differ from the outer diameter of tissue shaving instrument (100).

FIG. 8B shows adapter sheath (200) disposed on shaft assembly (120) of tissue shaving instrument (100). As shown in FIG. 8B, end effector (122) protrudes distally from shaft assembly (220) of adapter sheath (200) when adapter sheath (200) is disposed on shaft assembly (120) of tissue shaving instrument (100). Once adapter sheath (200) is positioned as desired relative to tissue shaving instrument (100), attachment portion (232) may be used to secure adapter sheath (200) to tissue shaving instrument (100). Securement of adapter sheath (200) first includes activating heating elements (250), as best seen in FIG. 8B. As described above, attachment portion (232) is configured to be responsive to heat or other environmental stimuli. Thus, as heating elements (250) increase in temperature, attachment portion (232) is configured to deform from the loose configuration shown in FIG. 8B to a tight configuration shown in FIG. 8C. In the present example, deformation includes a reduction in diameter of attachment portion (232). This reduction in diameter may continue until attachment portion (232) reaches an exact fit to the shape and size of outer shaft (130) of tissue shaving instrument (100). While hub (210) is positioned to engage hub (110) when adapter sheath (200) is secured to tissue shaving instrument (100) in this example, other scenarios may exist where hub (210) is not positioned to engage hub (110) when adapter sheath (200) is secured to tissue shaving instrument (100).

Once the tight configuration shown in FIG. 8C is reached, heating elements (250) may be deactivated such that heat is no longer generated. It should be understood that deactivation of heating elements (250) may be accomplished in a variety of ways. For instance, in some examples, deactivation may be accomplished automatically using one or more operational characteristics. By way of example only, one suitable operational characteristic may be temperature from one or more temperature sensors described above or from heating elements (250) themselves. Another suitable operational characteristic may be duration of heating, with each heating element (250) being automatically deactivated after a predetermined period of time. In other examples, combinations of operational characteristics may be used such as activation of a timer once a predetermined temperature is detected. In still other examples, deactivation of heating elements (250) may be achieved manually by an operator actuating a user input once a desired fit is achieved. In any case, the material forming attachment portion (232) may maintain the shrunk state after heat from heating elements (250) ceases, such that adapter sheath (200) remains secured to tissue shaving instrument (100) after heating elements (250) are deactivated.

### III. Example of an Alternative Navigation Adapter Sheath

As noted above, it may be desirable to incorporate structures similar to heating elements (250) into a structure similar to adapter sheath (200) in a variety of different configurations. Alternative configurations may be desirable to support flexibility in adapting structures similar to adapter sheath (200) to different instruments, procedures, operator preferences, etc. Thus, it should be understood that various alternative configurations of structures similar to heating elements (250) may be used while still encompassing the principles described herein.

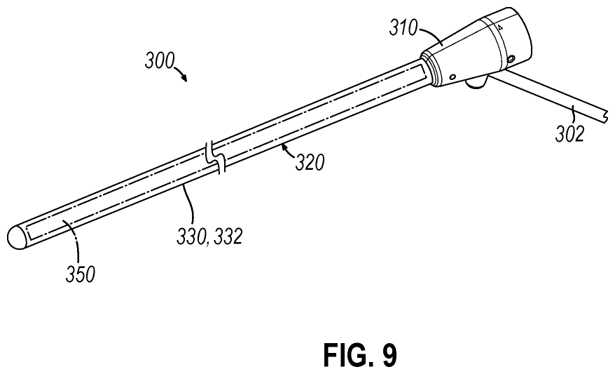

FIG. 9 shows an alternative adapter sheath (300), which includes one or more heating elements (350) in a different configuration relative to heating elements (250) described above. Unless otherwise noted herein, adapter sheath (300) of the present example is substantially similar to adapter sheath (250) described above. For instance, like adapter sheath (200), adapter sheath (300) of the present version includes a cable (302), a connector (not shown), an optional hub (310), and a shaft assembly (320) extending distally from the hub (310) and/or cable (302). Cable (302), the connector, and hub (310) are all substantially similar to corresponding structures described above such that similar details are omitted herein.

Shaft assembly (320) is similar to shaft assembly (220) described above in that shaft assembly (320) includes a hollow outer shaft (330), having open proximal and distal ends, that is configured to receive a shaft assembly of an instrument such as shaft assembly (120) of tissue shaving instrument (100) described above. Similarly, although not shown, shaft assembly (320) may be configured to receive a flex circuit similar to flex circuit (240) described above within the hollow interior of outer shaft (330). As similarly described above, the flex circuit is generally secured to the inner surface of outer shaft (330). Alternatively, in other examples, the flex circuit may be secured to the outer surface of outer shaft (330) or integrated into one or more portions of outer shaft (330).

Although not shown, it should also be understood that the flex circuit may likewise include structures similar to substrate (242), navigation sensors (244), traces (246, 252), and trace leads (248, 254) to facilitate electrical communication between IGS navigation system (50) and such navigation sensors and/or heating elements (350). The navigation sensors in the present version may be configured similarly to navigation sensors (244) described above with an electrically-conductive material forming a plurality of concentric loops.

The flex circuit of the present example also includes one or more heating elements (350). Unlike heating elements (250) described above, heating elements (350) of the present version are generally configured to extend along a greater length of outer shaft (330). In the present example, this length of heating elements (350) corresponds to about the entire length of outer shaft (330). Thus, the entirety or substantially all of outer shaft (330) may be characterized as an attachment portion (332). Although the length in present example is comparable to the length of outer shaft (330), it should be understood that in other examples, the length encompassed by heating elements (350) may be less than the length of outer shaft (330). Suitable alternative lengths may include one-half the length of outer shaft (330), three-quarters the length of outer shaft (330), and/or etc. Additionally, although each heating element (350) is shown as a single heating element (350) extending about the length of outer shaft (330), it should be understood that in other examples, multiple heating elements (350) may be combined to define the length of heating element (350) extension. In such multi-heating element (350) configurations, such heating elements (350) may be spaced from each other or positioned directly adjacent to each other.

Each heating element (350) of the present example is configured substantially similar to heating elements (250) described above. For instance, each heating element (350) includes a single loop of an electrically-conductive, metallic material such as copper. As described above, each single loop can include two generally parallel traces of metallic material serpentined within an area corresponding to the shape of at least a portion of attachment portion (332) and/or outer shaft (330). Alternatively, as similar described above, each heating element (350) may be configured in a multi-loop configuration rather than a single loop configuration.

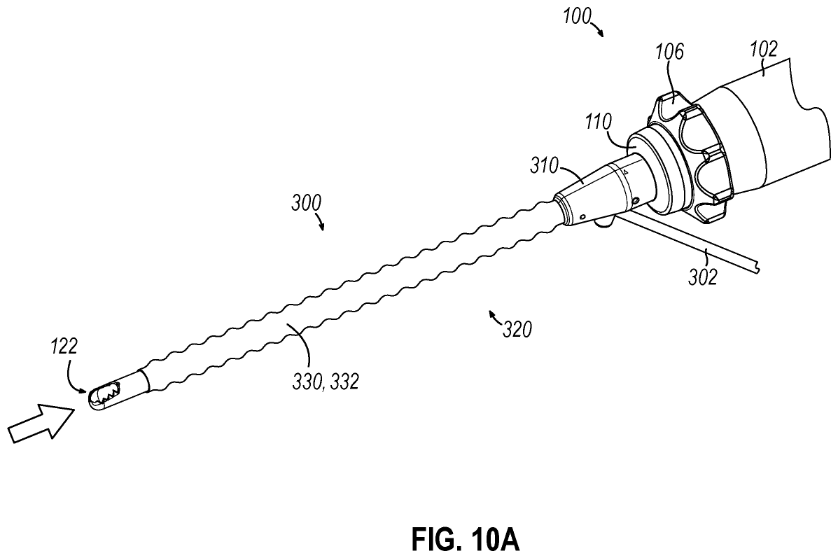

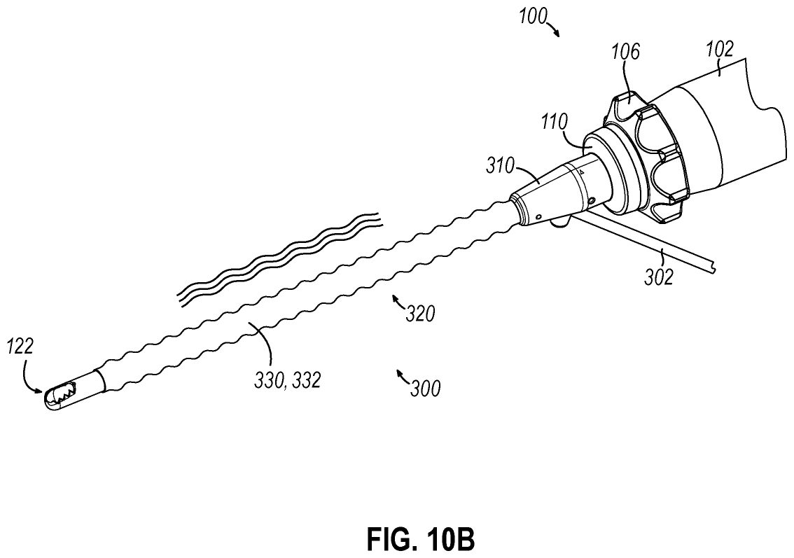

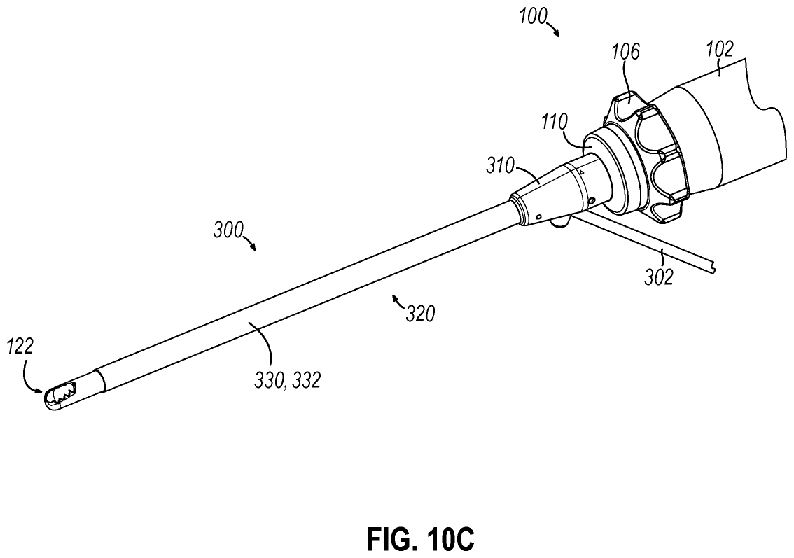

FIGS. 10A through 10C show an example of a use of adapter sheath (300) in combination with tissue shaving instrument (100). As with the use described above with respect to adapter sheath (200), adapter sheath (300) may be initially inserted onto shaft assembly (120) of tissue shaving instrument (100) or similar structures of any other suitable instrument. Also as with the use described above, adapter sheath (300) may be inserted onto shaft assembly (120) completely until hub (310) abuts hub (110). Alternatively, in some uses (such as versions where shaft assembly (120) is longer), adapter sheath (300) may only be partially inserted onto shaft assembly (120) such that a gap may be present between hub (310) and hub (110). Regardless of the particular degree of insertion, adapter sheath (300) may be inserted onto shaft assembly (120) to align relevant features of adapter sheath (300) (e.g., navigation sensors) with relevant features of tissue shaving instrument (100) (e.g., end effector (122)). As shown in FIG. 10A, end effector (122) protrudes distally from shaft assembly (320) of adapter sheath (300) when adapter sheath (300) is disposed on shaft assembly (120) of tissue shaving instrument (100).

After sliding of adapter sheath (300) onto shaft assembly (120) to a desired relative position, heating elements (350) may be activated as shown in FIG. 10B. Upon activation, heating elements (350) may heat the material of outer shaft (330), which is configured to respond to such heat to shrink from a loose configuration shown in FIG. 10B to a tight configuration shown in FIG. 10C. The loose configuration may provide at least some difference between the inner diameter of outer shaft (330) and the outer diameter of outer shaft (130) for case of insertion of adapter sheath (300) onto shaft assembly (120). Meanwhile, the tight configuration may substantially eliminate this clearance so that adapter sheath (300) may be secured to shaft assembly (120) with a substantially similar shape and substantially similar dimensions of outer shaft (130). In other words, outer shaft (330) may be deformed by heating elements (350) to fit outer shaft (130). This deformation includes a reduction in diameter of attachment portion (332) and/or outer shaft (330). This reduction in diameter may continue until attachment portion (332) and/or outer shaft (330) reaches an exact fit to the shape and size of outer shaft (130) of tissue shaving instrument (100).

Once the tight configuration shown in FIG. 10C is reached, heating elements (350) may be deactivated such that heat is no longer generated. It should be understood that deactivation of heating elements (350) may be accomplished in a variety of ways. For instance, in some examples, deactivation may be accomplished automatically using one or more operational characteristics. By way of example only, one suitable operational characteristic may be temperature from one or more temperature sensors described above or from heating elements (350) themselves. Another suitable operational characteristic may be duration of heating, with each heating element (350) being automatically deactivated after a predetermined period of time. In other examples, combinations of operational characteristics may be used such as activation of a timer once a predetermined temperature is detected. In still other examples, deactivation of heating elements (350) may be achieved manually by an operator actuating a user input once a desired fit is achieved. In any case, the material forming attachment portion (332) may maintain the shrunk state after heat from heating elements (350) ceases, such that adapter sheath (300) remains secured to tissue shaving instrument (100) after heating elements (350) are deactivated.

### IV. Examples of Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus for use with an ENT instrument, comprising: a shaft defining a hollow interior, the hollow interior being configured to receive a tubular portion of the ENT instrument; a heat shrink material integrated into a portion of the shaft; and a heating element fixedly secured to the shaft or heat shrink material, the heating element being configured to heat a portion of the heat shrink material to reduce a diameter of a portion of the shaft at a predetermined position along the length of the shaft.

### Example 2

The apparatus of Example 1, further comprising a navigation sensor secured to the shaft.

### Example 3

The apparatus of Example 2, the navigation sensor being positioned proximate a distal end of the shaft.

### Example 4

The apparatus of any of Examples 2 through 3, the navigation sensor being positioned proximate the heating element.

### Example 5

The apparatus of any of Examples 1 through 4, the heating element being positioned within the hollow interior of the shaft.

### Example 6

The apparatus of any of Examples 1 through 4, the heating element being integral with a portion of the shaft.

### Example 7

The apparatus of any of Examples 1 through 6, further comprising a flex-circuit having a flexible substrate, the heating element being defined by one or more electrically-conductive materials positioned on the flexible substrate.

### Example 8

The apparatus of Example 7, the electrically-conductive materials positioned on the flexible substrate defining a single loop including two parallel traces serpentined within an area corresponding to a shape of the heating element.

### Example 9

The apparatus of Example 7, the electrically-conductive materials positioned on the flexible substrate defining multiple loops, each loop including two parallel traces serpentined within an area corresponding to a shape of the heating element.

### Example 10

The apparatus of any of Examples 7 through 9, the flex-circuit being fixedly secured to the shaft.

### Example 11

The apparatus of any of Examples 7 through 10, the flex-circuit being configured to communicate with an IGS navigation system.

### Example 12

The apparatus of any of Examples 1 through 11, the heating element corresponding to a shape of at least a portion of the heat shrink material.

### Example 13

The apparatus of any of Examples 1 through 12, the heat shrink material being configured to transition between a loose configuration and a tight configuration, the heat shrink material being further configured to define a conical shape in the loose configuration.

### Example 14

The apparatus of any of Examples 1 through 12, the heat shrink material being configured to transition between a loose configuration and a tight configuration, the heat shrink material being further configured to define a substantially cylindrical shape in the loose configuration.

### Example 15

The apparatus of any of Examples 1 through 14, the heating element being in thermal communication with the heat shrink material.

### Example 16

The apparatus of any of Examples 1 through 15, the heating element being configured to generate signals indicating a real-time position of the heating element within three-dimensional space.

### Example 17

An apparatus, comprising: (a) a shaft having a distal end sized and configured to fit in an anatomical passageway of an ear, nose, or throat of a patient; (b) a flexible substrate extending along at least a portion of the shaft and fixedly secured thereto; (c) at least one navigation sensor positioned on the flexible substrate; and (d) at least one heating element positioned on the flexible substrate, the at least one heating element being configured to heat a portion of the shaft to reduce at least one dimension of the shaft.

### Example 18

The apparatus of Example 17, the flexible substrate including a first flexible substrate and a second flexible substrate, the first flexible substrate extending along the portion of the shaft opposite the second flexible substrate.

### Example 19

The apparatus of Example 18, the at least one navigation sensor including a first navigation sensor and a second navigation sensor, the first navigation sensor being positioned on the first flexible substrate and the second navigation sensor being positioned on the second flexible substrate, the at least one heating element including a first heating element and a second heating element, the first heating element being positioned on the first flexible substrate and the second heating element being positioned on the second flexible substrate.

### Example 20

The apparatus of any of Examples 17 through 18, shaft being configured to receive two or more different surgical instruments having different respective outer diameters.

### Example 21

A method of using an apparatus having (i) a substrate, (ii) at least one electrically-conductive navigation sensor trace formed on the substrate, and (iii) at least one heating element trace formed on the substrate, the method comprising: (a) positioning the substrate relative to a surgical instrument; (b) communicating power to the at least one heating element trace to generate heat from a portion of the substrate; (c) exposing the at least one navigation sensor trace to an electromagnetic field; (d) generating a signal via the at least one navigation sensor trace in response to the act of exposing the at least one navigation sensor trace to the electromagnetic field; and (e) determining location coordinates based on the generated signal.

### Example 22

The method of Example 21, further comprising shrinking at least a portion of a shaft secured to the substrate using the heat generated by the at least one heating element trace.

### Example 23

The method of Examples 21 or 22, the act of positioning the substrate relative to the surgical instrument including aligning the at least one navigation sensor trace with one or more features of the surgical instrument.

### Example 24

The method of any of Examples 21 through 23, further comprising stopping communication of electromagnetic energy to the at least one heating element trace prior to expositing the at least one navigation sensor trace to the electromagnetic field.

### Example 25

The method of any of Examples 21 through 24, further comprising exposing the at least one heating element trace to the electromagnetic field, and generating a signal via the at least one heating element trace in response to the act of exposing the at least one heating element trace to the electromagnetic field.

### Example 26

The method of Example 25, the act of determining location coordinates being based on the generated signal from the at least one navigation sensor trace and the generated signal from the at least one heating element trace.

### V. Miscellaneous

It should be understood that any of the teachings, expressions, embodiments, examples, etc. described herein may be combined with any of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus for use with an ENT instrument, comprising:
(a) a shaft defining a hollow interior, the hollow interior being configured to receive a tubular portion of the ENT instrument;
(b) a heat shrink material integrated into a portion of the shaft; and
(c) a heating element fixedly secured to the shaft or heat shrink material, the heating element being configured to heat a portion of the heat shrink material to reduce a diameter of a portion of the shaft at a predetermined position along the length of the shaft.

2. The apparatus of claim 1, further comprising a navigation sensor secured to the shaft.

3. The apparatus of claim 2, the navigation sensor being positioned proximate a distal end of the shaft.

4. The apparatus of claim 2, the navigation sensor being positioned proximate the heating element.

5. The apparatus of claim 1, the heating element being positioned within the hollow interior of the shaft.

6. The apparatus of claim 1, the heating element being integral with a portion of the shaft.

7. The apparatus of claim 1, further comprising a flex-circuit having a flexible substrate, the heating element being defined by one or more electrically-conductive materials positioned on the flexible substrate.

8. The apparatus of claim 7, the electrically-conductive materials positioned on the flexible substrate defining a single loop including two parallel traces serpentined within an area corresponding to a shape of the heating element.

9. The apparatus of claim 7, the electrically-conductive materials positioned on the flexible substrate defining multiple loops, each loop including two parallel traces serpentined within an area corresponding to a shape of the heating element.

10. The apparatus of claim 7, the flex-circuit being fixedly secured to the shaft.

11. The apparatus of claim 7, the flex-circuit being configured to communicate with an IGS navigation system.

12. The apparatus of claim 1, the heating element corresponding to a shape of at least a portion of the heat shrink material.

13. The apparatus of claim 1, the heat shrink material being configured to transition between a loose configuration and a tight configuration, the heat shrink material being further configured to define a conical shape in the loose configuration.

14. The apparatus of claim 1, the heat shrink material being configured to transition between a loose configuration and a tight configuration, the heat shrink material being further configured to define a substantially cylindrical shape in the loose configuration.

15. The apparatus of claim 1, the heating element being in thermal communication with the heat shrink material.

16. The apparatus of claim 1, the heating element being configured to generate signals indicating a real-time position of the heating element within three-dimensional space.

17. An apparatus, comprising:
(a) a shaft having a distal end sized and configured to fit in an anatomical passageway of an ear, nose, or throat of a patient;
(b) a flexible substrate extending along at least a portion of the shaft and fixedly secured thereto;
(c) at least one navigation sensor positioned on the flexible substrate; and
(d) at least one heating element positioned on the flexible substrate, the at least one heating element being configured to heat a portion of the shaft to reduce at least one dimension of the shaft.

18. The apparatus of claim 17, the flexible substrate including a first flexible substrate and a second flexible substrate, the first flexible substrate extending along the portion of the shaft opposite the second flexible substrate.

19. The apparatus of claim 18, the at least one navigation sensor including a first navigation sensor and a second navigation sensor, the first navigation sensor being positioned on the first flexible substrate and the second navigation sensor being positioned on the second flexible substrate, the at least one heating element including a first heating element and a second heating element, the first heating element being positioned on the first flexible substrate and the second heating element being positioned on the second flexible substrate.

20. A method of using an apparatus having (i) a substrate, (ii) at least one electrically-conductive navigation sensor trace formed on the substrate, and (iii) at least one heating element trace formed on the substrate, the method comprising:
(a) positioning the substrate relative to a surgical instrument;
(b) communicating power to the at least one heating element trace to generate heat from a portion of the substrate;
(c) exposing the at least one navigation sensor trace to an electromagnetic field;
(d) generating a signal via the at least one navigation sensor trace in response to the act of exposing the at least one navigation sensor trace to the electromagnetic field; and
(e) determining location coordinates based on the generated signal.
